(19) United States
(12) Patent Application Publication
(10) Pub. No.: US 2018/0303961 A1
(43) Pub. Date: Oct. 25, 2018

(54) CARDIAC ULTRAVIOLET SANITIZER

(71) Applicants: Jacob DeLaRosa, Pocatello, ID (US); Michael Jackson, Pocatello, ID (US); Juan Leon, Pocatello, ID (US)

(72) Inventors: Jacob DeLaRosa, Pocatello, ID (US); Michael Jackson, Pocatello, ID (US); Juan Leon, Pocatello, ID (US)

(21) Appl. No.: 15/497,137

(22) Filed: Apr. 25, 2017

(51) Int. Cl. A61L 2/10; A61L 2/00

(52) U.S. Cl. CPC A61L 2/10; A61L 2202/11; A61L 2202/122; A61L 2/0023

(57) ABSTRACT

A sterilization apparatus for use with a cardioplegia heat exchanger device includes an enclosure having an internal volume defined between two ends and a wall. The first end includes a bulb access port, an inlet port formed proximate to or in the first end, a biofilm sterilizing port, and an outlet port formed proximate to or in the second end. A quartz sleeve and an ultraviolet bulb project inwardly into the internal volume from proximate the bulb access port. The ultraviolet bulb illuminates to radiate water entering the inlet port and flowing towards the outlet port between the cylindrical wall and the quartz sleeve. A wiper ring may be slidably fixed around the quartz sleeve for cleaning. In an alternate embodiment, the bulb access port is formed through a water tank of the cardioplegia heat exchanger device with the quartz sleeve and bulb projecting inwardly into the water tank.

### CROSS-REFERENCE TO RELATED APPLICATIONS

This application is a continuation-in-part of U.S. patent application Ser. No. 15/333,173, filed on Oct. 24, 2016, and is incorporated herein by reference.

### STATEMENT REGARDING FEDERALLY SPONSORED RESEARCH AND DEVELOPMENT

Not Applicable.

### FIELD OF THE INVENTION

This invention relates to sterilization apparatuses, and more particularly to a device for sterilizing water in a cardiac heater/cooler apparatus.

### DISCUSSION OF RELATED ART

Certain medical devices, such as cardioplegia heat exchangers and the like, include water the circulates from a water tank, through the device, and back into the water tank. While such water is treated in a way to regulate its temperature, for example, such devices typically have no means for readily sterilizing the water against viruses, molds, bacterial contamination, and biofilm.

Therefore, there is a need for a device that sterilizes the water in medical devices, such as cardioplegia heat exchangers, against viruses, molds, bacterial contamination, and biofilm formation, and indeed brings such water up to potable water quality. Such a needed invention would be usable within a surgical arena, and would be easily adapted for use with existing cardioplegia heat exchanger devices. Such a needed device would be simple to maintain and cost effective to use. The present invention accomplishes these objectives.

### SUMMARY OF THE INVENTION

The present device is a sterilization apparatus for use with a cardioplegia heat exchanger device. A cylindrical enclosure of the sterilization apparatus has a first end, an opposing second end, and a cylindrical wall between the ends. An internal volume is defined within the cylindrical enclosure between the first end, the second end, and the cylindrical wall. The first end includes a bulb access port, an inlet port formed proximate to or in the first end, and an outlet port formed proximate to or in the second end.

A quartz sleeve, substantially transparent to UV light, projects inwardly into the internal volume from proximate the bulb access port. An ultraviolet bulb projects inwardly within the quartz sleeve from the bulb access port, protected within a watertight seal of the quartz sleeve with the enclosure.

As such, the ultraviolet bulb is connected with a power source. The ultraviolet bulb illuminates to radiate any water entering the inlet port and flowing towards the outlet port between the cylindrical wall and the quartz sleeve. The ultraviolet light sterilizes the water for use within the cardioplegia heat exchanger device. An ozone port is further include for introducing ozone or other sterilizing agents into the internal volume.

In some embodiments, the sterilization apparatus further includes at least one mounting bracket fixed with the cylindrical enclosure, such that the sterilization apparatus is mountable to the cardioplegia heat exchanger device with at least one mechanical fastener.

Preferably the sterilization apparatus further includes a sleeve wiper accept port formed through the second end that is selectively sealable with a port seal cap. A quartz sleeve wiper ring is slidably fixed within the internal volume and adapted to fit around the quartz sleeve. The quartz sleeve wiper ring includes at least one resilient wiper blade for contacting the quartz sleeve. The quartz sleeve wiper ring is selectively fixable with a plunger rod fixed thereto through the access port for manually sliding the quartz sleeve wiper ring from one end of the quartz sleeve to another end of the quartz sleeve, thereby cleaning the quartz sleeve of any deposits that may have formed on its surface during regular use.

In an alternate embodiment of the invention, the sterilization apparatus is used with a cardioplegia heat exchanger device of the type having a water tank fixed within a heat exchanger housing. In such an embodiment the bulb access port is formed through the water tank with the quartz sleeve projecting inwardly into the water tank from proximate the bulb access port. The ultraviolet bulb projects inwardly within the quartz sleeve from the bulb access port. As such, with the ultraviolet bulb connected with the power source to illuminate the bulb, ultraviolet light radiates water entering the water tank, thereby sterilizing the water within the cardioplegia heat exchanger device. Such an embodiment does not require a separate enclosure, as the water tank of the cardioplegia heat exchanger device replaces such an enclosure.

It has been found that adding an additional sterilizing agent within the sterilization apparatus, such as ozone, enhances the sterilizing effect of water circulating throughout the device and of any biofilm buildup, biofilm being any type of bacteria buildup that adheres to a surface. Our previous U.S. patent application Ser. No. 15/333,173, filed on Oct. 24, 2016, did not include such an auxiliary sterilizing agent generator or a biofilm sterilizing port, which in the instant application can be included proximate the first end of the cylindrical enclosure.

The present invention sterilizes the water in medical devices, such as cardioplegia heat exchangers, against viruses, molds and bacterial contamination, and indeed brings such water up to potable water quality. The present device is usable within a surgical arena, and is easily adapted for use with existing cardioplegia heat exchanger devices. The present invention is simple to maintain and cost effective to use. Other features and advantages of the present invention will become apparent from the following more detailed description, taken in conjunction with the accompanying drawings, which illustrate, by way of example, the principles of the invention.

### DESCRIPTION OF THE DRAWINGS

FIG. 1 is side elevational view of the invention;

FIG. 2 is a cross-sectional view bisecting the invention vertically along its longitudinal axis; and

FIG. 3 is a diagram of the invention as installed in a cardioplegia heat exchanger device.

### DETAILED DESCRIPTION OF THE PREFERRED EMBODIMENT

Illustrative embodiments of the invention are described below. The following explanation provides specific details for a thorough understanding of and enabling description for these embodiments. One skilled in the art will understand that the invention may be practiced without such details. In other instances, well-known structures and functions have not been shown or described in detail to avoid unnecessarily obscuring the description of the embodiments.

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively. Additionally, the words “herein,” “above,” “below” and words of similar import, when used in this application, shall refer to this application as a whole and not to any particular portions of this application. When the claims use the word “or” in reference to a list of two or more items, that word covers all of the following interpretations of the word: any of the items in the list, all of the items in the list and any combination of the items in the list. When the word “each” is used to refer to an element that was previously introduced as being at least one in number, the word “each” does not necessarily imply a plurality of the elements, but can also mean a singular element.

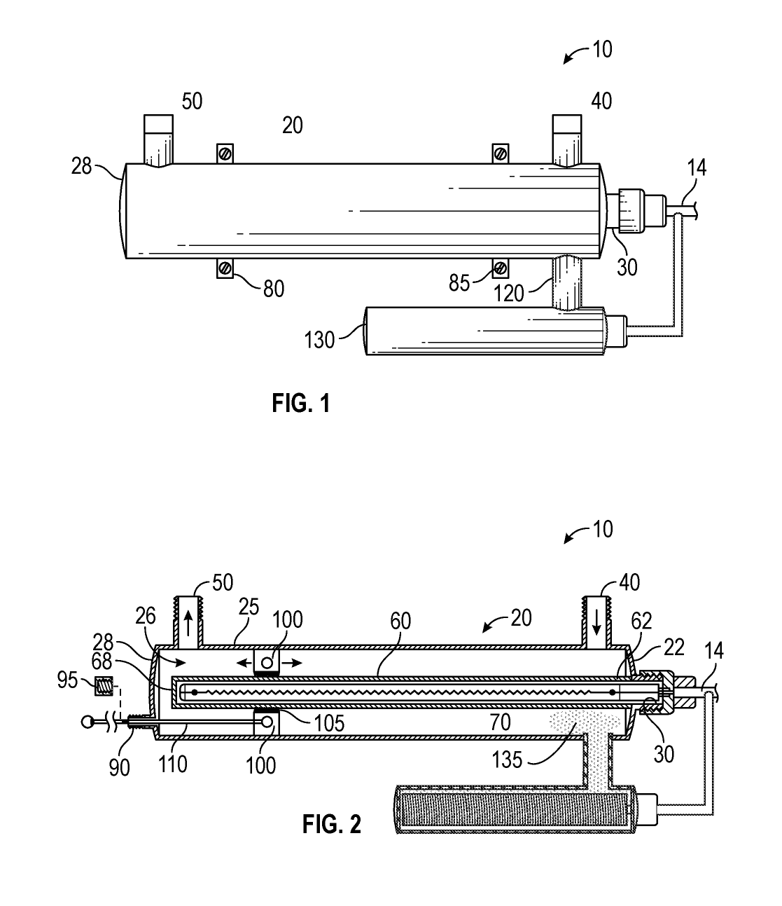

FIGS. 1 and 2 illustrate a sterilization apparatus 10 for use with a water-circulating device 15, such as a cardioplegia heat exchanger device 15. For example, the cardioplegia heat exchanger device 15 may be a Sorin 3T brand Heater-Cooler System, or the like. Such devices 15 are design to regulate patient temperature during certain surgeries by heating water that is recirculated to the patient.

A cylindrical enclosure 20 of the sterilization apparatus 10 has a first end 22, an opposing second end 28, and a cylindrical wall 25 between the ends 22,28. An internal volume 26 is defined within the cylindrical enclosure 20 between the first end 22, the second end 28, and the cylindrical wall 25. The first end 22 includes a bulb access port 30, an inlet port 40 formed proximate to or in the first end 22, and an outlet port 50 formed proximate to or in the second end 28. Alternately, the inlet port 40 may be formed proximate to or in the second end 28 with the outlet port 50 formed proximate to or in the first end 22. The cylindrical enclosure 20 may be made from a rigid material such as aluminum, stainless steel, strong injection-molded plastic, or the like.

A quartz sleeve 60, substantially transparent to UV light, projects inwardly into the internal volume 26 from proximate the bulb access port 30. An ultraviolet bulb 70 projects inwardly within the quartz sleeve 60 from the bulb access port 30, protected within a watertight seal of the quartz sleeve 60 with the enclosure 20. The ultraviolet bulb 70 may be removed from the quartz sleeve 60 through the bulb access port 30 to replace the ultraviolet bulb 70 when necessary, any water 19 within the enclosure 20 prevented from exiting the bulb access port 30 by the quartz sleeve 60 and its seal with the enclosure 20.

As such, the ultraviolet bulb 70 is connected with a power source 14, such as a power cord connected to a wall outlet, a battery, or the like. The ultraviolet bulb 70, preferably in the 15-55 watt range, illuminates to radiate any water 19 entering the inlet port 40 and flowing towards the outlet port 50 between the cylindrical wall 25 and the quartz sleeve 60. The ultraviolet light sterilizes the water 19 for use within the cardioplegia heat exchanger device 15, preferably delivering ultraviolet C radiation within a wavelength range of 230-270 nanometers with an intensity of at least 45,000 MW/cm² so as to sterilize viruses, which is approximately three times the intensity necessary to sterilize bacteria.

In some embodiments, the sterilization apparatus 10 further includes at least one mounting bracket 80 fixed with the cylindrical enclosure 20, such that the sterilization apparatus 10 is mountable to the cardioplegia heat exchanger device 15 with at least one mechanical fastener 85. Typically, the sterilization apparatus 10 is connected to an inlet and outlet of both a hot cardioplegia tank (not shown) and a main patient tank, allowing for the water 19 to circulate through the apparatus 10 when pumps (not shown) of the cardioplegia heat exchanger device 15 are activated. Once all of the water 19 is sterilized, the cardioplegia heat exchanger device 15 may be enabled for standard use.

Preferably the sterilization apparatus 10 further includes a sleeve wiper accept port 90 formed through the second end 28 that is selectively sealable with a port seal cap 95. A quartz sleeve wiper ring 100 (FIG. 2) is slidably fixed within the internal volume 26 and adapted to fit around the quartz sleeve 60. The quartz sleeve wiper ring 100 includes at least one resilient wiper blade 105 for contacting the quartz sleeve 60. The quartz sleeve wiper ring 100 is selectively fixable with a plunger rod 110 fixed thereto through the access port 90 for manually sliding the quartz sleeve wiper ring 100 from one end 62 of the quartz sleeve 60 to another end 68 of the quartz sleeve 60, thereby cleaning the quartz sleeve 60 of any deposits that may have formed on its surface during regular use.

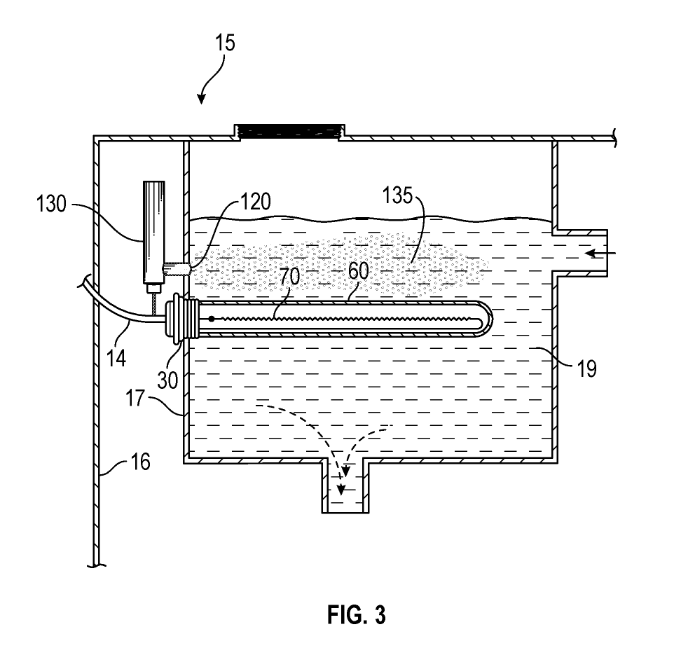

In an alternate embodiment of the invention, the sterilization apparatus 10 is used with a cardioplegia heat exchanger device 15 of the type having a water tank 17 (FIG. 3) fixed within a heat exchanger housing 16. In such an embodiment the bulb access port 30 is formed through the water tank 17 with the quartz sleeve 60 projecting inwardly into the water tank 17 from proximate the bulb access port 30, the quartz sleeve 60 typically being oriented either horizontally or vertically within the water tank 17. The ultraviolet bulb 70 projects inwardly within the quartz sleeve 60 from the bulb access port 30. As such, with the ultraviolet bulb connected with the power source 14 to illuminate the bulb 70, ultraviolet light radiates water 19 entering the water tank 17, thereby sterilizing the water 19 within the cardioplegia heat exchanger device 15. Such an embodiment does not require a separate enclosure 20, as the water tank 17 of the cardioplegia heat exchanger device 15 replaces such an enclosure 20.

In the present invention the cylindrical enclosure 20 further includes a biofilm sterilizing port 120 formed proximate the first end 22 thereof. As such a sterilizing agent 135 may be introduced into the biofilm sterilizing port 120 to further sterilize the water 19. Preferably the sterilizing agent 135 is ozone produced by an ozone generator 130 fixed with the biofilm sterilizing port 120 and electrically connected to the power source 14, either with a separate power cord or combined with the power cord powering the bulb 70.

While a particular form of the invention has been illustrated and described, it will be apparent that various modifications can be made without departing from the spirit and scope of the invention. For example, the shape of the enclosure 20 may be adapted to fit the shape of the quartz sleeve 60 and bulb 70. Likewise, the device 10 could be applied to a wide variety of other medical devices having circulating water, even, for example, a hot water bottle for use under a patient. Accordingly, it is not intended that the invention be limited, except as by the appended claims.

Particular terminology used when describing certain features or aspects of the invention should not be taken to imply that the terminology is being redefined herein to be restricted to any specific characteristics, features, or aspects of the invention with which that terminology is associated. In general, the terms used in the following claims should not be construed to limit the invention to the specific embodiments disclosed in the specification, unless the above Detailed Description section explicitly defines such terms. Accordingly, the actual scope of the invention encompasses not only the disclosed embodiments, but also all equivalent ways of practicing or implementing the invention.

The above detailed description of the embodiments of the invention is not intended to be exhaustive or to limit the invention to the precise form disclosed above or to the particular field of usage mentioned in this disclosure. While specific embodiments of, and examples for, the invention are described above for illustrative purposes, various equivalent modifications are possible within the scope of the invention, as those skilled in the relevant art will recognize. Also, the teachings of the invention provided herein can be applied to other systems, not necessarily the system described above. The elements and acts of the various embodiments described above can be combined to provide further embodiments.

All of the above patents and applications and other references, including any that may be listed in accompanying filing papers, are incorporated herein by reference. Aspects of the invention can be modified, if necessary, to employ the systems, functions, and concepts of the various references described above to provide yet further embodiments of the invention.

Changes can be made to the invention in light of the above “Detailed Description.” While the above description details certain embodiments of the invention and describes the best mode contemplated, no matter how detailed the above appears in text, the invention can be practiced in many ways. Therefore, implementation details may vary considerably while still being encompassed by the invention disclosed herein. As noted above, particular terminology used when describing certain features or aspects of the invention should not be taken to imply that the terminology is being redefined herein to be restricted to any specific characteristics, features, or aspects of the invention with which that terminology is associated.

While certain aspects of the invention are presented below in certain claim forms, the inventor contemplates the various aspects of the invention in any number of claim forms. Accordingly, the inventor reserves the right to add additional claims after filing the application to pursue such additional claim forms for other aspects of the invention.

What is claimed is:

1. A sterilization apparatus for use with a water-circulating device, the sterilization apparatus comprising:
a cylindrical enclosure having a first end, an opposing second end, and a cylindrical wall between the ends, an internal volume defined therebetween, the first end including a bulb access port, a biofilm sterilizing port formed proximate the first end, an inlet port formed proximate the first end, an outlet port formed proximate the second end;
a quartz sleeve projecting inwardly into the internal volume from proximate the bulb access port;
an ultraviolet bulb projecting inwardly within the quartz sleeve from the bulb access port;
whereby with a sterilizing agent introduced into the biofilm sterilizing port, and with the ultraviolet bulb connected with a power source to illuminate the bulb, ultraviolet light radiates water entering the inlet port and flowing to the outlet port between the cylindrical wall and the quartz sleeve, thereby sterilizing the water for use within the water-circulating device, the sterilizing agent further sanitizes the water and any biofilm in contact therewith.

2. The sterilization apparatus of claim 1 further including at least one mounting bracket fixed with the cylindrical enclosure, whereby the sterilization apparatus is mountable to the water-circulating device with at least one mechanical fastener.

3. The sterilization apparatus of claim 1 wherein the inlet port is formed within the first end.

4. The sterilization apparatus of claim 1 wherein the inlet port is formed within the cylindrical wall proximate the first end.

5. The sterilization apparatus of claim 1 wherein the outlet port is formed within the second end.

6. The sterilization apparatus of claim 1 wherein the outlet port is formed within the cylindrical wall proximate the second end.

7. The sterilization apparatus of claim 1 further including a sleeve wiper access port formed through the second end, the sleeve wiper access port selectively sealable with a port seal cap, a quartz sleeve wiper ring included within the internal volume and adapted to fit around the quartz sleeve, the quartz sleeve wiper ring including at least one resilient wiper blade for contacting the quartz sleeve, the quartz sleeve wiper ring selectively fixable with a plunger rod fixed thereto through the access port for manually sliding the quarts sleeve wiper ring from one end of the quartz sleeve to the other end of the quartz sleeve.

8. The sterilization apparatus of claim 1 wherein the water-circulating device is a cardioplegia heat exchanger device.

9. The sterilization apparatus of claim 1 wherein the sanitizing agent is ozone.

10. The sterilization apparatus of claim 9 wherein an ozone generator is fixed with the biofilm sterilizing port.

11. The sterilization apparatus of claim 10 wherein the ozone generator is electrically powered and electrically connected with the power source.

12. A sterilization apparatus for use with a water-circulating device of the type having a water tank fixed within a heat exchanger housing, the sterilization apparatus comprising:
a bulb access port formed into the water tank;
a quartz sleeve projecting inwardly into the water tank from proximate the bulb access port;
an ultraviolet bulb projecting inwardly within the quartz sleeve from the bulb access port; and
a biofilm sterilizing port formed proximate the first end;
whereby with the ultraviolet bulb connected with a power source to illuminate the bulb, ultraviolet light radiates water entering the water tank, thereby sterilizing the water within the water-circulating device, and with a sanitizing agent introduced into the biofilm sterilizing port the sanitizing agent further sterilizes the water and disrupts and eliminates biofilm.

13. The sterilization apparatus of claim 12 wherein the water-circulating device is a cardioplegia heat exchanger device.

14. The sterilization apparatus of claim 12 wherein the sanitizing agent is ozone.

15. The sterilization apparatus of claim 14 wherein an ozone generator is fixed with the biofilm sterilizing port.

16. The sterilization apparatus of claim 15 wherein the ozone generator is electrically powered and electrically connected with the power source.
